(19) United States
(12) Patent Application Publication
(10) Pub. No.: US 2017/0281988 A1
(43) Pub. Date: Oct. 5, 2017

(54) Skin Care and Cosmetic Composition

(71) Applicants: Zhiyun WU, Guangzhou (CN); Chungwah MA, Guangzhou (CN); Shiyu ZOU, Guangzhou (CN)

(72) Inventors: Zhiyun WU, Guangzhou (CN); Chungwah MA, Guangzhou (CN); Shiyu ZOU, Guangzhou (CN)

(73) Assignee: Infinitus (China) Company Ltd., Jiangmen (CN)

(21) Appl. No.: 14/258,031

(22) Filed: Apr. 22, 2014

(51) Int. Cl. A61Q 19/08; A61K 8/92; A61K 8/64; A61K 8/02; A61K 8/06

(52) U.S. Cl. CPC A61Q 19/08; A61K 8/022; A61K 8/06; A61K 8/64; A61K 8/92

(57) ABSTRACT

A skin care composition and product having beneficial effects on skin conditions/symptoms, such as reducing wrinkles and skin roughness and improving skin elasticity, etc. The composition has three separate parts: a powder part, an aqueous part and an emulsion part. The power part comprises Oligopeptide-1 and Ginseng saponin. The aqueous part comprises Glycyrrhiza glabra Root Extract, Artemisia capillaris Flower Extract, Radix Mori Albae Extract, Zizyphus jujuba Fruit Extract, Scutellaria baicalensis Root Extract, hydrolyzed rice protein and nicotinamide. The emulsion part comprises Bifida ferment lysate, creatine, carnosine, Glucosyl Hesperidin, Hexapeptide-3, Centella asiatica Extract, Coenzyme Q10 capsule, Opuntia Ficus-indica Stem Extract, Rhodiola rosea Extract, Saussurea Involucrata Extract, Panax notoginseng Root Extract and Angelica sinensis Extract capsule.

### FIELD OF THE INVENTION

The present invention related to the cosmetic field, in particular it relates to a skin care composition and product with beneficial cosmetic effects and/or effects of relieving common undesirable skin conditions or symptoms.

### BACKGROUND OF THE INVENTION

Skin aging is part of the aging process of human body, which not only relates to the age itself, but also closely relates to sunlight exposure. The light induced skin aging (“light-aging”) is mainly caused by sunlight irradiation, and as depletion of the ozone layer is aggravating, the light-aging of the skin becomes more and more serious, which affects not only the appearance, but may also cause underlying etiological problems relating to skin cancer.

Extracts of traditional herbal medical materials used in China (“TCM”) generally comprises many active ingredients, such as saponins, flavonoids, and alkaloids, many of which ingredients have various skin-care functions. Beauty and skin-care products based on herbal ingredients have thousands of years of history in China. Particularly, nowadays, people prefer natural substances rather than chemically synthesized substances. Therefore, herbal medicine cosmetic products are getting more popular.

### SUMMARY OF THE INVENTION

An object of the present invention is to provide a skin care composition with beneficial cosmetic effects and/or effects of relieving common skin symptoms. The object is achieved by the following technical solution: selecting active ingredients for their effects of regulating immunity and for their effects in delaying skin aging process induced by sunlight and formulating a skin care composition comprising a powder part, an aqueous part and an emulsion part. The ingredients for each part and the rationale for their selection are summarized in the following.

The powder part comprises the following ingredients in portion by weight:

| | |
|---|---|
| Oligopeptide-1 | 0~1.0 portion |
| Ginseng saponin | 0~5.0 portions |

and at least one of the two ingredients must be present.

The aqueous part comprises the following ingredients in portion by weight:

| | |
|---|---|
| Glycyrrhiza glabra Root Extract | 0~3.0 portions |
| Artemisia capillaris Flower Extract | 0~3.0 portions |
| Radix Mori Albae Extract | 0~2.0 portions |
| Zizyphus jujuba Fruit Extract | 0~2.0 portions |
| Scutellaria baicalensis Root Extract | 0~2.0 portions |
| hydrolyzed rice protein | 0~5.0 portions |
| nicotinamide | 0~3.0 portions |

and at least one of the ingredients must be present.

The emulsion part comprises the following ingredients in portion by weight:

| | |
|---|---|
| Bifida ferment lysate | 0.5~10.0 portions |
| creatine | 0~2.0 portions |
| carnosine | 0~2.0 portions |
| Glucosyl Hesperidin | 0~5.0 portions |
| Hexapeptide-3 | 0~5.0 portions |
| Centella asiatica Extract | 0~3.0 portions |
| Coenzyme Q10 capsule | 0~2.0 portions |
| Opuntia Ficus-indica Stem Extract | 0~5.0 portions |
| Rhodiola rosea Extract | 0~2.0 portions |
| Saussurea Involucrata Extract | 0~5.0 portions |
| Panax notoginseng Root Extract | 0~5.0 portions |
| Angelica sinensis Extract capsule | 0~5.0 portions |

and, in addition to Bifida ferment lysate, one of the other ingredients must be present.

In the powder part, Oligopeptide-1 is an epidermal growth factor, which is a protein secreted by microorganism in a high density and highly purified form, and has a biological effect of promoting the growth and metabolism of skin epidermal cells. Ginseng saponin might be the total saponins extracted and processed from the root of Panax plant Panax ginseng C. A. Mey, or may be any monomer of Ginseng saponin or the mixture of various monomers of Ginseng saponin, such as the mixture of one or two of Ginseng saponin Rb1, Rb2, Rb3, Rc, Rd, Rg3 and Rh2.

In the aqueous formulation, hydrolyzed rice protein is the zymolyte obtained through the co-hydrolysis of rice protein by alkaline protease and complex protease, which comprises thymic penta peptide. It can prevent the UV-induced atrophy of langerhans cells, prevent the UV-induced change of skin structure and integrity, protect the skin structure and integrity thereof, and promote the synthesis of β1-integrin, keratin and filaggrin, so that the immune activity of skin is enhanced. It is proved by the culture experiment of human keratinocytes that the hydrolyzed rice protein can improve cellular vitality by 34%.

Glycyrrhiza glabra Root Extract comprises Glabridin, an isoflavones component, whose chemical structure comprises the nucleus of isoflavone, with ring A and ring B as aromatic rings, and ring B comprising 2 phenolic hydroxyl groups, and an ethylene bond on C9, therefore has a rather strong anti-oxidation activity. Rhodiola rosea has an effect on immune regulation, and it prevents the UV-caused fibroblast apoptosis, and delays the light-aging of skin.

The emulsion part of the present invention may be lotion, gel and cream formulations, where, Bifida ferment lysate is the lysate solution of Bifidobacterium obtained by means of bio-technique, which comprises metabolic product, cytoplasmic component, cell wall component and polysaccharide complex. It is found in studies that UV irradiation will reduce the secretion of IL-12, affect the proliferation of NK cells and T cells, increase the expression of IL-10 and cause immunosuppression, and thereby render DNA damage. It is proved by experiments that Bifida ferment lysate can fight against the immune suppression caused by the UV-induced increase of IL-10 expression, stimulate the natural repair mechanism (matrix) of skin DNA after the UV irradiation, and postpone the UV-caused aging of skin.

Creatine can accumulate the stored energy of cells, thereby increasing the available ATP, and promote DNA repair and the DNA synthesis of cells after UV irradiation injury, which reduces the sunburn of cells induced by UV obviously and postpones the aging process of skin.

Carnosine, also termed as dipeptide or β-alanyl-L-histidine, has the effects of removing active free radicals, against oxidation, against glycosylation reaction, against protein cross-linking, chelating heavy metal ions, and postponing the shortening process of telomere, thus postpones the aging of skin.

Hesperidin is a kind of natural biological flavonoids extracted from citrus peel and termed as “Vitamin P”, it has the effects of strengthening capillary resistance, reducing permeability and anti-inflammation. Glucosyl Hesperidin is a derivative of water-soluble Hesperidin, whose water solubility is increased by enzymatic combination techniques. It is proved by experiments that Glucosyl Hesperidin has the effects of enhancing blood circulation, improving skin tone and brightness.

Hexapeptide-3 is homologous substance having a 6-amino acid sequence in the Type-III unit of fibronectin protein molecule. It is proved by experiments that Hexapeptide-3 can improve cellular adhesion and the expression of β1-integrin, facilitate a more even cellular extension on collagen, thus contribute to the prevention of light aging of skin.

Centella asiatica Extract comprises the active components, being total triterpene of Centella asiatica and madecassoside, which have the effects of promoting the proliferation of human fibroblasts in the skin, promoting the synthesis of collagen and inhibiting inflammation. Centella asiatica Extract can promote the regeneration of aged skin and promote the healing of wound.

Coenzyme Q10 is an anti-oxidant, which is the activator of cellular respiration and metabolism, it can bring energy and vitality to the body and skin, and can scavenge the active oxygen from the body. When the skin is affected externally or internally, a large number of free radicals are produced during the inflammatory procedure, whereas Coenzyme Q10 can remove free radicals and postpone skin aging. It is proved by experiments that, by improving the activity of Cathepsin D, stratum corneum chymotrypsin (SCCE) and stratum corneum trypsin-like enzymes (SCTE), Opuntia Ficus-indica Stem Extract can promote the shedding of keratinocytes, accelerate cell renewal, improve the transparency and gloss of skin, and reduce fine lines and wrinkles.

Saussurea Involucrata Extract comprises the active components of polysaccharide and flavone, it has the effects of promoting and regulating immune function, as well as fighting against free radicals. Together with other polysaccharide components and fibrous protein in the skin, it can form extracellular gel matrix comprising a large amount of moisture to moisturize the skin, delay the aging problems of skin, such as drying and loss of elasticity, etc. The active component of Panax notoginseng Root is Notoginseng total saponin, it has an obvious effect on improving the skin SOD activity, reducing MDA content and improving the hydroxyproline in the skin.

Angelica sinensis is the dried Root of Umbrelliferae plant Angelica sinensis, it is able to eliminate free radicals, complex ferrous ion and inhibit tyrosinase, and has the effects of preventing skin aging and promoting skin whitening. Angelica sinensis Root mainly comprises volatile oil and non-volatile oil components, with heavy odor. Through encapsulation, the odor is reduced; meanwhile, the active ingredient are made easily permeable.

The Coenzyme Q10 capsule refers to the Coenzyme Q10 encapsulated by means of conventional liposome encapsulation techniques.

Preferably, the weight portions of the components of the powder part are:

| | |
|---|---|
| Oligopeptide-1 | 0.01~0.5 portion |
| Ginseng saponin | 0.1~2.0 portions. |

More preferably, the weight portions of the components of the powder part are:

| | |
|---|---|
| Oligopeptide-1 | 0.01~0.05 portion |
| Ginseng saponin | 0.10~0.50 portion. |

Preferably, the weight portions of the components of the aqueous part are:

| | |
|---|---|
| Glycyrrhiza glabra Root Extract | 0.10~1.50 portions |
| Artemisia capillaris Flower Extract | 0.10~1.50 portions |
| Radix Mori Albae Extract | 0.10~1.00 portion |
| Zizyphus jujuba Fruit Extract | 0.10~1.00 portion |
| Scutellaria baicalensis Root Extract | 0.10~1.00 portion |
| hydrolyzed rice protein | 0.10~2.00 portions |
| nicotinamide | 0.10~1.50 portions. |

More preferably, the weight portions of the components of the aqueous part are:

| | |
|---|---|
| Glycyrrhiza glabra Root Extract | 0.5~1.0 portion |
| Artemisia capillaris Flower Extract | 0.5~1.0 portion |
| Radix Mori Albae Extract | 0.5~1.0 portion |
| Zizyphus jujuba Fruit Extract | 0.5~1.0 portion |
| Scutellaria baicalensis Root Extract | 0.5~1.0 portion |
| hydrolyzed rice protein | 0.5~1.0 portion |
| nicotinamide | 0.50~1.0 portion. |

Preferably, the weight portions of the components of said emulsion part are:

| | |
|---|---|
| Rhodiola rosea Extract | 0.1~2.0 portions |
| Bifida ferment lysate | 0.5~5.0 portions |
| creatine | 0.5~1.5 portions |
| carnosine | 0.1~1.0 portion |
| Glucosyl Hesperidin | 0.1~2.0 portion |
| Hexapeptide-3 | 0.5~1.5 portions |
| Centella asiatica Extract | 0.1~1.0 portion |
| Coenzyme Q10 capsule | 0.1~1.0 portion |
| Opuntia Ficus-indica Stem Extract | 0.1~3.0 portions. |

Besides the aforesaid major ingredients, the powder part, aqueous part and emulsion part according to the present invention may also include the common matrix for the external dosage form of cosmetics. They include excipients such as mannitol, disodium hydrogen phosphate and sodium dihydrogen phosphate. The common matrix for aqueous formulation comprises excipients such as sodium hyaluronate, oxhide glue, butanediol, disodium EDTA, dipotassium glycyrrhetate, panthenol and preservatives. The common matrix for emulsion formulation comprises excipients such as bisabolol, cetylhydroxyproline palmitamide, brassica campestris sterol, jojoba seed oil, phytosterol isostearate, Vitamin E acetate, silicone oil, Cetyl stearyl alcohol, Sucrose polystearate, Beheneth-25, Dipalmitoyl hydroxyproline, Acrylates/C10-30 alkyl acrylate crosspolymer, butanediol, glycerin, Sodium stearoyl glutamate, disodium EDTA, xanthan gum, deionized water, acrylamide/ammonia acrylate copolymer (and) Poly(isobutylene) (and) Polysorbate-20, 1-methylhydantoin-2-imide, preservatives.

The present invention has the following beneficiary effects:

(1). The combined administration of the three parts of the product has an effect of reducing wrinkles, reducing skin roughness, fading uneven skin tone, improving skin elasticity, firming skin, making the skin more delicate and looking younger.

(2) The product can produce its effects quickly, and only requires intermittent administration, e.g., an administration of 28 days (one period) in 3 months, or it is administered when the skin condition is unsatisfying.

(3) The separation of the powder part, aqueous part and emulsion part of the present invention ensures the active components intact during the storage process, thereby ensuring their effects and function when used.

### BRIEF DESCRIPTION OF THE DRAWINGS

FIG. 1 shows the overall effect of the skin care product of two particular embodiments on volunteers participated in a trial in comparison with a comparative products available in the prior art.

FIG. 2 shows the effects on specific skin conditions in the same trial as FIG. 2 of two particular embodiments and a comparative products available in the prior art.

The various features of novelty which characterize the invention are pointed out with particularity in the claims annexed to and forming a part of this disclosure. For a better understanding of the invention, its operating advantages, and specific objects attained by its use, reference should be made to the drawings and the following description in which there are illustrated and described preferred embodiments of the invention.

### DETAILED DESCRIPTION OF THE INVENTION

The present invention will be further described as follows in combination with specific embodiments, but the embodiments shall not restrict the present invention in any forms. The material and ingredients used in the embodiments are all commercially available. Unless specifically addressed, the portion number of each ingredient in the following description is weight portion.

### Embodiment 1

### 1. Preparation of the Powder Part:

The ingredients are:

| | |
|---|---|
| Oligopeptide-1 | 0.1 portion; |
| Ginseng saponin | 1.0 portion; |
| mannitol | 10.0 portions; |
| disodium hydrogen phosphate | 0.5 portion; |
| sodium dihydrogen phosphate | 0.2 portion. |

The preparation process is as follows:

(1) Dissolve all the components in an appropriate amount of deionized water, and filter the resulting solution through a 0.2 μm membrane.

(2) Fill it into small glass vials, and place the vials into a vacuum freeze drier.

(3) Vacuum dry for 45 hours so that it is in the form of powder.

### 2. Preparation of Aqueous Part:

The ingredients are:

| | | |
|---|---|---|
| Glycyrrhiza glabra Root Extract | 2.0 | portions; |
| Artemisia capillaris Flower Extract | 2.0 | portions; |
| Radix Mori Albae Extract | 1.0 | portion; |
| Zizyphus jujuba Fruit Extract | 1.0 | portion; |
| Scutellaria baicalensis Root Extract | 1.0 | portion; |
| hydrolyzed rice protein | 3.0 | portions; |
| nicotinamide | 2.0 | portions; |
| sodium hyaluronate | 0.5 | portion; |
| butanediol | 10.0 | portions; |
| disodium EDTA | 0.5 | portion; |
| dipotassium glycyrrhetate | 1.0 | portion; |
| panthenol | 5.0 | portions; |
| preservatives | 0.1 | portion; |

| | |
|---|---|
| deionized water | in an appropriate amount. |

The molecular weight of the sodium hyaluronate is 300,000.

The preparation process is as follows:

(1) Add sodium hyaluronate to the deionized water, and stir to dissolve.

(2) Add all other components and stir to dissolve.

(3) Adjust the resulting solution to pH 5.5˜7.0, and passing quality examination fill it into small vials.

### 3. Preparation of a Lotion Part

The ingredients are:

### Phase A:

| | |
|---|---|
| jojoba seed oil | 8 portions; |
| phytosterol isostearate | 5 portions; |
| Vitamin E acetate | 3 portions; |
| Rhodiola rosea Extract | 0.1 portion; |
| Palmitoyl hydrolyzed wheat protein | 3 portions; |
| Cetyl stearyl alcohol | 2 portions; |
| Sucrose polystearate | 2 portions; |
| hydrogenated Poly(isobutylene) | 5 portions; |
| Dipalmitoyl hydroxyproline | 2 portions; |

### Phase B:

| | | |
|---|---|---|
| Acrylates/C10-30 alkyl acrylate crosspolymer | 0.5 | portion; |
| butanediol | 10 | portions; |
| glycerin | 5 | portions; |
| Sodium stearoyl glutamate | 1 | portion; |
| disodium EDTA | 0.1 | portion; |
| xanthan gum | 0.5 | portion; |

| | |
|---|---|
| deionized water | margin; |

### Phase C:

acrylamide/ammonia acrylate copolymer (and) Poly(isobutylene) 2.0 portions; (and) Polysorbate-20

### Phase D:

| | |
|---|---|
| aminomethyl propanol | 0.2 portion |
| 1-methylhydantoin-2-imide | 1.0 portion; |
| creatine | 0.5 portion; |
| carnosine | 0.5 portion; |
| Glucosyl Hesperidin | 1.0 portion; |
| dipotassium glycyrrhetate | 2.0 portions; |
| Bifida ferment lysate | 5.0 portions; |
| Hexapeptide-3 | 0.5 portion |
| Centella asiatica Extract | 1.0 portion; |
| Coenzyme Q10 capsule | 1.0 portion; |
| Opuntia Ficus-indica Stem Extract | 2.0 portions; |
| Saussurea Involucrata Extract | 0.5 portion; |
| Panax notoginseng Root Extract | 0.5 portion; |
| Angelica sinensis Extract capsule | 0.5 portion; |
| preservatives | 0.1 portion. |

The preparation process is as follows:

(1) All components in Phase A are mixed evenly, heated to 80° C., stirred to dissolve to obtain Phase A solution;

(2) Acrylates/C10-30 alkyl acrylate crosspolymer is added to deionized water, stirred to dissolve, then other raw materials in Phase B are added, heated to 80° C., stirred to dissolve to obtain Phase B solution;

(3) Phase A solution is added to Phase B solution, and then all components in Phase C are added, stirred to homogenize to be emulsified;

(4) The mixture is stirred and cooled to 45° C., aminomethyl propanol is added, stirred to dissolve, and then other raw materials in Phase D are added, stirred and cooled to 30˜35° C.;

(5) A lotion is obtained, whose pH value is adjusted to 5.5˜6.5, upon passing quality examination, filled to the vessels.

### Embodiment 2

### 1. Preparation of Powder Part

The ingredients are:

| | |
|---|---|
| Oligopeptide-1 | 0.5 portion; |
| Ginseng saponin | 2.0 portions; |
| mannitol | 15 portions; |
| disodium hydrogen phosphate | 0.5 portion; |
| sodium dihydrogen phosphate | 0.2 g portion. |

The preparation process is as follows:

(1) All components are dissolved in an appropriate amount of deionized water, then the obtained solution is filtered through a 0.2 μm membrane.

(2) The solution is loaded on to a plate which is put into a vacuum freeze drier.

(3) The solution is being vacuum dried for 45 hours into a form of powder, which is then distributed into small vials.

### 2. Preparation of Aqueous Part

The ingredients are:

| | |
|---|---|
| Glycyrrhiza glabra Root Extract | 0.5 portion |
| Artemisia capillaris Flower Extract | 0.5 portion |
| Radix Mori Albae Extract | 0.5 portion |
| Zizyphus jujuba Fruit Extract | 0.5 portion |
| Scutellaria baicalensis Root Extract | 0.5 portion |
| hydrolyzed rice protein | 1.0 portion |
| sodium hyaluronate | 0.5 portion |
| butanediol | 8.0 portions |
| disodium EDTA | 0.1 portion |
| dipotassium glycyrrhetate | 0.5 portion |
| panthenol | 0.5 portion |
| nicotinamide | 0.3 portion |
| xanthan gum | 1.2 portions |
| preservatives | 0.1 portion |
| deionized water | in an appropriate amount. |

The molecular weight of the sodium hyaluronate is 300,000.

The preparation process is as follows:

(1) Xanthan gum is added to the deionized water, stirred to dissolve.

(2) All the other components are added to the solution of (1), stirred to dissolve.

(3) An aqueous formulation is obtained, which is adjusted to PH to 5.5˜6.5, and after passing the quality examination, filled into vessels.

### 3. Preparation of a Cream

The ingredients are:

### Phase A:

| | | |
|---|---|---|
| jojoba seed oil | 5 | portions |
| phytosterol isostearate | 2 | portions |
| Vitamin E acetate | 5 | portions |
| Rhodiola rosea Extract | 0.5 | portion |
| Cetyl stearyl alcohol | 3 | portions |
| Sucrose polystearate | 5 | portions |
| Dipalmitoyl hydroxyproline | 2 | portions |

### Phase B:

| | | |
|---|---|---|
| Acrylates/C10-30 alkyl acrylate crosspolymer | 1.5 | portions |
| butanediol | 15 | portions |
| glycerin | 15 | portions |
| Sodium stearoyl glutamate | 0.5 | portion |
| disodium EDTA | 0.1 | portion |
| xanthan gum | 0.5 | portion |

| | |
|---|---|
| deionized water | margin |

### Phase C:

acrylamide/ammonia acrylate copolymer (and) Poly(isobutylene) 2.0 portions; (and) Polysorbate-20

### Phase D:

| | | |
|---|---|---|
| aminomethyl propanol | 0.15 | portion |
| 1-methylhydantoin-2-imide | 1.0 | portion |
| creatine | 0.5 | portion |
| carnosine | 0.5 | portion |
| Glucosyl Hesperidin | 1.0 | portion |
| dipotassium glycyrrhetate | 2.0 | portions |
| Bifida ferment lysate | 5.0 | portions |
| Hexapeptide-3 | 1.0 | portion |
| Centella asiatica Extract | 1.0 | portion |
| Coenzyme Q10 capsule | 1.0 | portion |
| Opuntia Ficus-indica Stem Extract | 2.0 | portions |
| Saussurea Involucrata Extract | 0.5 | portion |
| Panax notoginseng Root Extract | 0.5 | portion |
| Angelica sinensis Extract capsule | 0.5 | portion |
| preservatives | 0.1 | portion |

The preparation is as follows:

(1) The components in Phase A are heated to 80˜90° C., stirred to dissolve.

(2) Acrylates/C10-30 alkyl acrylate crosspolymer is added to deionized water, stirred to dissolve, then other raw materials in Phase B are added, heated to 80˜90° C., stirred to dissolve.

(3) Phase A is added to Phase B, stirred to homogenize to be emulsified, and meanwhile, the raw materials in Phase C are added;

(4) The above mixture is stirred and cooled to 45° C., and aminomethyl propanol is added, stirred to dissolve, and then other raw materials in Phase D are added, stirred and cooled to 30˜35° C.;

(5) A cream is obtained, whose pH value is adjusted to 5.5˜7.0;

(6) Passing quality examination, the cream is then filled into vessels.

### Example 3

### 1. Preparation of Powder Part

The ingredients are:

| | |
|---|---|
| Oligopeptide-1 | 0.5 portion |
| Ginseng saponin | 2.0 portions |
| mannitol | margin; |

The preparation process is as follows:

(1) All components are mixed evenly.

(2) The mixture are divided into small portions and filled into sachets.

### 2. Preparation of Aqueous Part

The ingredients are:

| | | |
|---|---|---|
| Glycyrrhiza glabra Root Extract | 2.0 | portions |
| Artemisia capillaris Flower Extract | 2.0 | portions |
| Radix Mori Albae Extract | 1.0 | portion |
| Zizyphus jujuba Fruit Extract | 1.0 | portion |
| Scutellaria baicalensis Root Extract | 1.0 | portion |
| hydrolyzed rice protein | 3.0 | portions |
| glycerin | 10.0 | portions |
| disodium EDTA | 0.1 | portion |
| dipotassium glycyrrhetate | 2.0 | portions |
| panthenol | 0.5 | portion |
| nicotinamide | 1.0 | portion |
| Acrylates/C10-30 alkyl acrylate crosspolymer | 1.0 | portion |
| aminomethyl propanol | 0.15 | portion |
| preservatives | 0.10 | portion |

| | |
|---|---|
| deionized water | in an |
| | appropriate |
| | amount. |

The molecular weight of the sodium hyaluronate is 300,000.

The preparation process is as follows:

(1) Acrylates/C10-30 alkyl acrylate crosspolymer is added to the deionized water, stirred to disperse, aminomethyl propanol is added, stirred to dissolve completely.

(2) All the other components are added to (1), stirred to dissolve.

(3) Adjusted pH to 5.5˜7.0.

(4) Upon passing quality control, fill it into vessels.

### 3. Preparation of a Gel

The ingredient are:

### Phase A:

| | | |
|---|---|---|
| Cetyl stearyl alcohol | 2 | portions |
| Sucrose polystearate | 2 | portions |
| Dipalmitoyl hydroxyproline | 2 | portions |
| Rhodiola rosea Extract | 0.1 | portion |

### Phase B:

Acrylates/C10-30 alkyl acrylate crosspolymer 3 portions

| | |
|---|---|
| butanediol | 10 portions |
| glycerin | 5 portions |
| disodium EDTA | 0.1 portion |
| xanthan gum | 0.5 portion |
| deionized water | margin |

### Phase C:

acrylamide/ammonia acrylate copolymer (and) Poly(isobutylene) 2.0 portions; (and) Polysorbate-20

### Phase D:

| | |
|---|---|
| aminomethyl propanol | 0.3 portion |
| creatine | 0.5 portion |
| carnosine | 0.5 portion |
| Glucosyl Hesperidin | 1.0 portion |
| dipotassium glycyrrhetate | 2.0 portions |
| Bifida ferment lysate | 5.0 portions |
| Hexapeptide-3 | 0.5 portion |
| Centella asiatica Extract | 1.0 portion |
| Coenzyme Q10 capsule | 1.0 portion |
| Opuntia Ficus-indica Stem Extract | 2.0 portions |
| Saussurea Involucrata Extract | 0.5 portion |
| Panax notoginseng Root Extract | 0.5 portion |
| Angelica sinensis Extract capsule | 0.5 portion |
| preservatives | 0.1 portion. |

The preparation process is as follows:

(1) The raw materials in Phase A are heated to 80˜90° C., stirred to dissolve;

(2) Acrylates/C10-30 alkyl acrylate crosspolymer is added to deionized water, stirred to dissolve, then other raw materials in Phase B are added, heated to 80˜90° C., stirred to dissolve.

(3) Phase A is added to Phase B, stirred to homogenize to be emulsified, and meanwhile, the raw materials in Phase C are added;

(4) The above mixture is stirred and cooled to 45° C., aminomethyl propanol is added, stirred to dissolve, and then other raw materials in Phase D are added, stirred and cooled to 30˜35° C.;

(5) A gel is obtained, whose pH value is adjusted to 5.5˜7.0;

(6) Upon passing examination, the gel is then filled into vessels.

### Example 4

### 1. Preparation of Powder Part

The ingredients are:

| | | |
|---|---|---|
| Oligopeptide-1 | 0.001 | portion; |
| Ginseng saponin | 0.1 | portion; |
| mannitol | 5.0 | portions; |
| disodium hydrogen phosphate | 0.5 | portion; |
| sodium dihydrogen phosphate | 0.2 | portion. |

The preparation process is as follows:

(1) All components are dissolved in an appropriate amount of deionized water, then the obtained solution is sterilized in an autoclave at 121° C., 0.1 Mpa for 30 mins.

(2) It is distributed into small glass vials, and then place them into a vacuum freeze drier.

(3) It is then vacuum dried for 45 hours into the form of powder, and then vials are capped.

### 2. Preparation of Aqueous Part

The ingredients are:

| | | |
|---|---|---|
| Glycyrrhiza glabra Root Extract | 0.5 | portion; |
| Artemisia capillaris Flower Extract | 0.5 | portion; |
| Radix Mori Albae Extract | 0.3 | portion; |
| Zizyphus jujuba Fruit Extract | 0.3 | portion; |
| Scutellaria baicalensis Root Extract | 0.3 | portion; |
| hydrolyzed rice protein | 1.0 | portion; |
| nicotinamide | 0.5 | portion; |
| sodium hyaluronate | 0.1 | portion; |
| butanediol | 8.0 | portions; |
| disodium EDTA | 0.1 | portion; |
| dipotassium glycyrrhetate | 0.1 | portion; |
| panthenol | 0.5 | portion; |
| preservatives | 0.01 | portion; |

| | |
|---|---|
| deionized water | in an appropriate amount. |

The molecular weight of the sodium hyaluronate is 300,000.

The preparation process is as follows:

(1) Sodium hyaluronate is added to the deionized water, stirred to dissolve.

(2) Other components are added, stirred to dissolve.

(3) An aqueous part is obtained, whose pH value is adjusted to 5.5˜7.0, and upon passing quality control, filled into vials.

### 3. Preparation of a Lotion

The ingredients are:

### Phase A:

| | | |
|---|---|---|
| phytosterol isostearate | 0.5 | portion; |
| Vitamin E acetate | 1 | portion; |
| Cetyl stearyl alcohol | 1 | portion; |
| Sucrose polystearate | 2 | portions; |
| Dipalmitoyl hydroxyproline | 1 | portion; |
| Rhodiola rosea Extract | 0.1 | portion |

### Phase B:

| | | |
|---|---|---|
| Acrylates/C10-30 alkyl acrylate crosspolymer | 0.1 | portion; |
| butanediol | 5 | portions; |
| glycerin | 2 | portions; |
| Sodium stearoyl glutamate | 0.5 | portion; |
| disodium EDTA | 0.1 | portion; |
| xanthan gum | 0.1 | portion; |

| | |
|---|---|
| deionized water | margin; |

### Phase C:

acrylamide/ammonia acrylate copolymer (and) Poly(isobutylene) 1.0 portion; (and) Polysorbate-20

### Phase D:

| | |
|---|---|
| aminomethyl propanol | 0.2 portion |
| 1-methylhydantoin-2-imide | 1.0 portion; |
| creatine | 0.2 portion; |
| carnosine | 0.2 portion; |
| Glucosyl Hesperidin | 0.5 portion; |
| dipotassium glycyrrhetate | 0.1 portion; |
| Bifida ferment lysate | 1.0 portion; |
| Centella asiatica Extract | 0.5 portion; |
| Coenzyme Q10 capsule | 0.5 portion; |
| Opuntia Ficus-indica Stem Extract | 1.0 portion; |
| Hexapeptide-3 | 1.0 portion; |
| Saussurea Involucrata Extract | 0.1 portion; |
| Panax notoginseng Root Extract | 0.1 portion; |
| Angelica sinensis Extract capsule | 0.1 portion; |
| preservatives | 0.05 portion. |

The preparation process is as follows:

(1) All components in Phase A are mixed evenly, heated to 80° C., stirred to dissolve to obtain Phase A solution.

(2) Acrylates/C10-30 alkyl acrylate crosspolymer is added to deionized water, stirred to dissolve, then other raw materials in Phase B are added, heated to 80° C., stirred to dissolve to obtain Phase B solution.

(3) Phase A solution is added to Phase B solution, and then all components in Phase C are added, stirred to homogenize to be emulsified.

(4) The above mixture is stirred and cooled to 45° C., aminomethyl propanol is added, stirred to dissolve, and then other raw materials in Phase D are added, stirred and cooled to 30˜35° C.;

(5) A lotion is obtained, whose pH value is adjusted to 5.5˜6.5, and upon passing quality control, filled into the vessels.

### Example 5

### 1. Preparation of Powder Part

The ingredients are:

| | |
|---|---|
| Oligopeptide-1 | 1.0 portion; |
| Ginseng saponin | 5.0 portions; |
| mannitol | 20.0 portions. |

The preparation process is as follows:

(1) All components are dissolved in an appropriate amount of deionized water, then the obtained solution is sterilized in an autoclave at 121° C., 0.1 Mpa for 30 mins.

(2) Distribute it into glass vials, and then put into the vacuum freeze drier.

(3) Vacuum freeze dried for 45 hours, to form a powder, then cap the vials.

### 2. Preparation of Aqueous Part

The ingredients are:

| | |
|---|---|
| Glycyrrhiza glabra Root Extract | 3.0 portions; |
| Artemisia capillaris Flower Extract | 3.0 portions; |
| Radix Mori Albae Extract | 2.0 portions; |
| Zizyphus jujuba Fruit Extract | 2.0 portions; |
| Scutellaria baicalensis Root Extract | 2.0 portions; |
| hydrolyzed rice protein | 5.0 portions; |
| nicotinamide | 3.0 portions; |
| sodium hyaluronate | 2.0 portions; |
| butanediol | 20.0 portions; |
| disodium EDTA | 0.1 portion; |
| dipotassium glycyrrhetate | 2.0 portions; |
| panthenol | 5.0 portions; |
| preservatives | 0.01 portion; |
| deionized water | in an appropriate amount. |

The molecular weight of the sodium hyaluronate is 300,000.

The preparation process is as follows:

(1) Sodium hyaluronate is added to the deionized water, stirred to dissolve.

(2) Other components are added, stirred to dissolve;

(3) An aqueous part is obtained, whose pH value is adjusted to 5.5˜7.0, upon passing quality examination, filled into vessels.

### 3. Preparation of a Lotion

The ingredients are:

### Phase A:

| | |
|---|---|
| phytosterol isostearate | 0.5 portion; |
| Vitamin E acetate | 3 portions; |
| Rhodiola rosea Extract | 2.0 portions |
| Cetyl stearyl alcohol | 1 portion; |
| Sucrose polystearate | 2 portions; |
| Dipalmitoyl hydroxyproline | 1 portion; |

### Phase B:

Acrylates/C10-30 alkyl acrylate crosspolymer 0.1 portion;

| | |
|---|---|
| butanediol | 5 portions; |
| glycerin | 2 portions; |
| Sodium stearoyl glutamate | 0.5 portion; |
| disodium EDTA | 0.1 portion; |
| xanthan gum | 0.1 portion; |
| deionized water | margin; |

### Phase C:

acrylamide/ammonia acrylate copolymer (and) Poly(isobutylene) 1.0 portion; (and) Polysorbate-20

### Phase D:

| | |
|---|---|
| aminomethyl propanol | 0.2 portion; |
| 1-methylhydantoin-2-imide | 1.0 portion; |
| creatine | 2.0 portions; |
| carnosine | 2.0 portions; |
| Glucosyl Hesperidin | 5.0 portions; |
| dipotassium glycyrrhetate | 0.1 portion; |
| Bifida ferment lysate | 10.0 portions; |
| Hexapeptide-3 | 5.0 portions |
| Centella asiatica Extract | 3.0 portions; |
| Coenzyme Q10 capsule | 0.5 portion; |
| Opuntia Ficus-indica Stem Extract | 1.0 portion; |
| Saussurea Involucrata Extract | 0.1 portion; |
| Panax notoginseng Root Extract | 0.1 portion; |
| Angelica sinensis Extract capsule | 0.1 portion; |
| preservatives. | 0.05 portion. |

The preparation process as follows:

(1) All components in Phase A are mixed evenly, heated to 80° C., stirred to dissolve to obtain Phase A solution.

(2) Acrylates/C10-30 alkyl acrylate crosspolymer is added to deionized water, stirred to dissolve, then other raw materials in Phase B are added, heated to 80° C., stirred to dissolve to obtain Phase B solution.

(3) Phase A solution is added to Phase B solution, and all components in Phase C are added, stirred to homogenize to be emulsified.

(4) The above mixture is stirred and cooled to 45° C., aminomethyl propanol is added, stirred to dissolve, and then other components in Phase D are added, stirred and cooled to 30˜35° C.;

(5) A lotion is obtained, whose pH value is adjusted to 5.5˜6.5, upon passing quality examination, the lotion filled in vessels.

### Comparative Product

The comparative product used in the present invention is a skin care product available in the prior art, which has a composition as follows (portions by weight):

| | |
|---|---|
| PPG-3 Benzyl ether myristate | 2~5 portions |
| Polyglycerin-3 methylglucose distearate | 2~5 portions |
| Hydrogenated Poly(isobutylene) | 2~5 portions |
| Palmitoyl Pentapeptide-3 | 2~5 portions |
| Polyglycerin-3 methylglucose distearate | 1~3 portions |
| Oat Extract | 1~3 portions |
| hexadecanol - octadecanol | 1~3 portions |
| Glycerin monosterate | 1~3 portions |
| bisabolol | 0.1~0.5 portion |
| Butcher's Broom Root Extract | 0.1~0.3 portion |
| Centella asiatica Extract | 0.1~0.5 portion |
| panthenol | 0.1~0.5 portion |
| Calendula officinalis L. Extract | 0.1~0.5 portion |
| Hydrolyzed yeast protein | 0.1~0.5 portion |
| Aesculus hippocastanum Extract | 0.1~0.5 portion |
| Monoammonium glycyrrhizinate | 0.1~0.5 portion |
| BOSWELLIA SERRATA Extract | 0.1~0.3 portion |
| carnosine | 0.1~0.5 portion |
| xanthan gum | 0.1~0.5 portion |
| Radix Ginseng Extract | 0.1~0.5 portion |
| Ganoderma lucidum Extract | 0.1~0.5 portion |
| Boswellia carteri Extract | 0.1~0.5 portion |
| Oligopeptide-1 | 0.1~0.5 portion |
| preservatives | 0.1~0.2 portion |

For all the embodiments described above, the three parts of the composition are separately prepared and stored in separate containers until being used by users. In use, first mix the powder part in the aqueous part in a weight ratio between 1:50 and 1:500, preferably 1:100, and apply the mixture to the skin under gentle massage. Then, the emulsion part (in an amount roughly equal to the mixture of the power and aqueous part) is applied to the skin. The product is to be applied twice a day in the morning and the evening, respectively.

### Testing Conducted and Effects Observed

In order to confirm the intended effects of the present invention, testing was conducted on female volunteers who had various undesirable skin conditions or symptoms and voluntarily enrolled for the trial. For each particular skin conditions (see below), three groups of volunteers, with 50 or more in each group, were assigned to use the products of embodiment 1, embodiment 2 and comparative prior art (described above), respectively. For each of the following skin conditions, the products were applied to the facial skin twice a day for 4 weeks (about 3% volunteers terminated the use due to side effects during the 4-week trial period). The application of the product on facial skin was conducted in a manner that a person normally uses a cosmetic product. About one gram of the product was used each time with a ratio of 1:100:100 among the power part, aqueous part and emulsion part (first applying the mixture of power and aqueous parts and then applying the emulsion part with a gentle massage in between). At the end of each week during the trial, each volunteer was asked about what was her degree of satisfaction with the product on a scale from 1 to 5 and how effective she found the product on relieving a particular skin condition on a scale from 1 to 3 (1 means no effect, 2 means some effect, and 3 means significant effect). The following skin conditions/symptoms were tried:

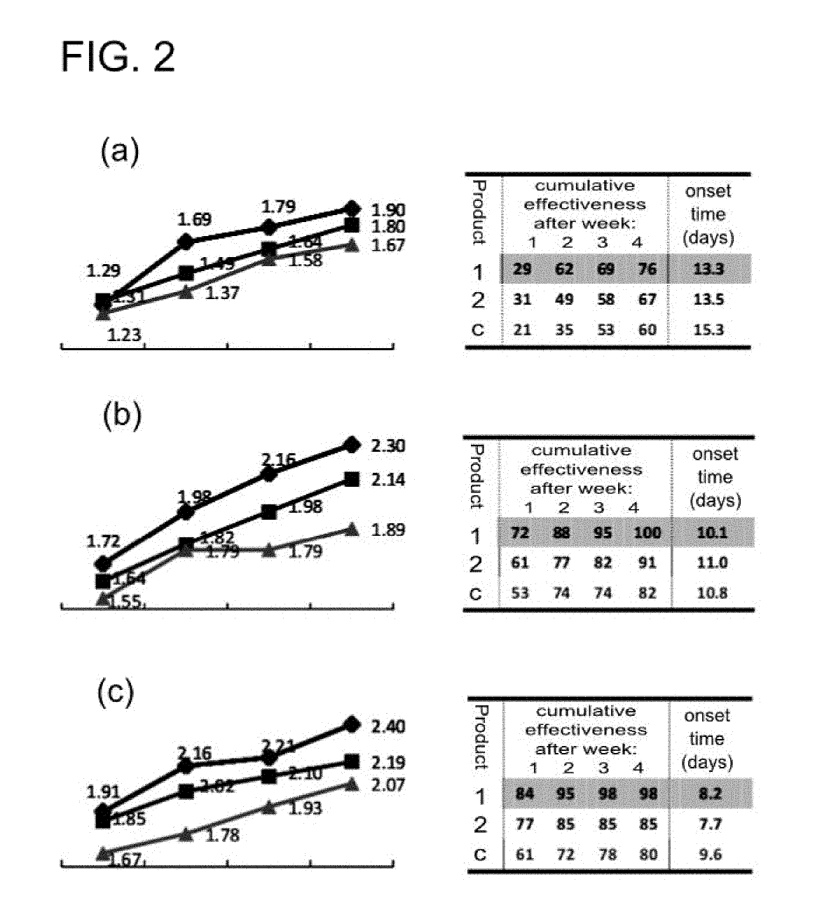

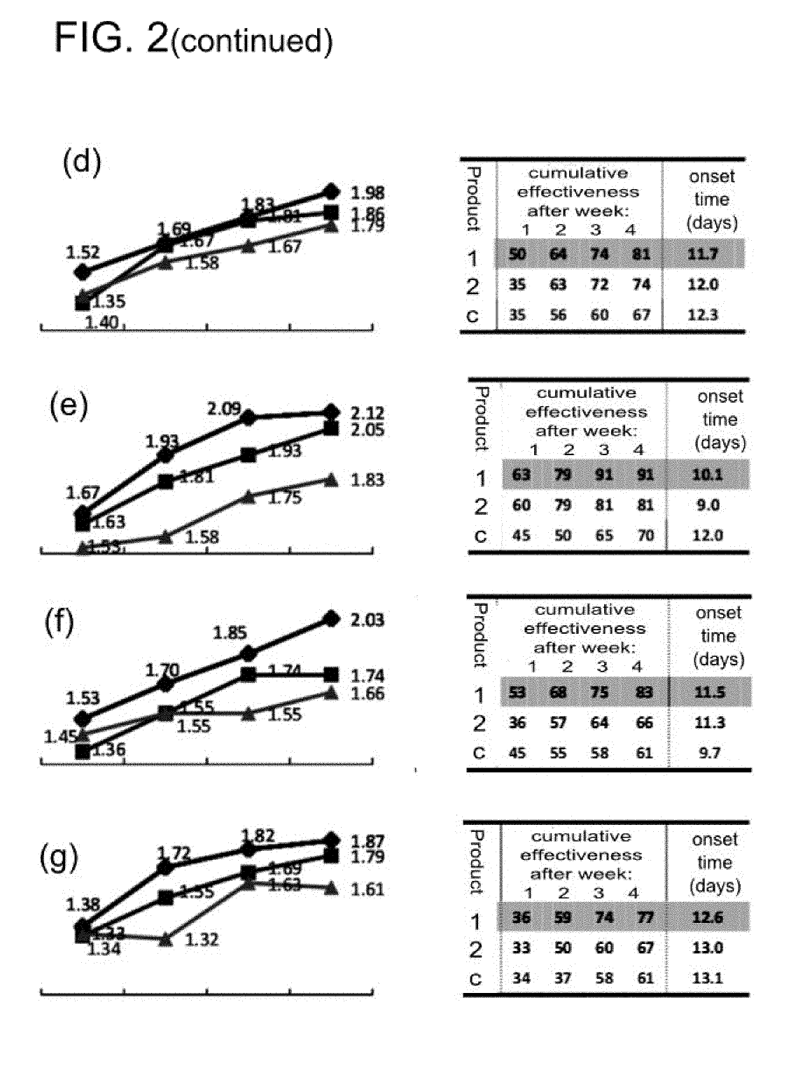

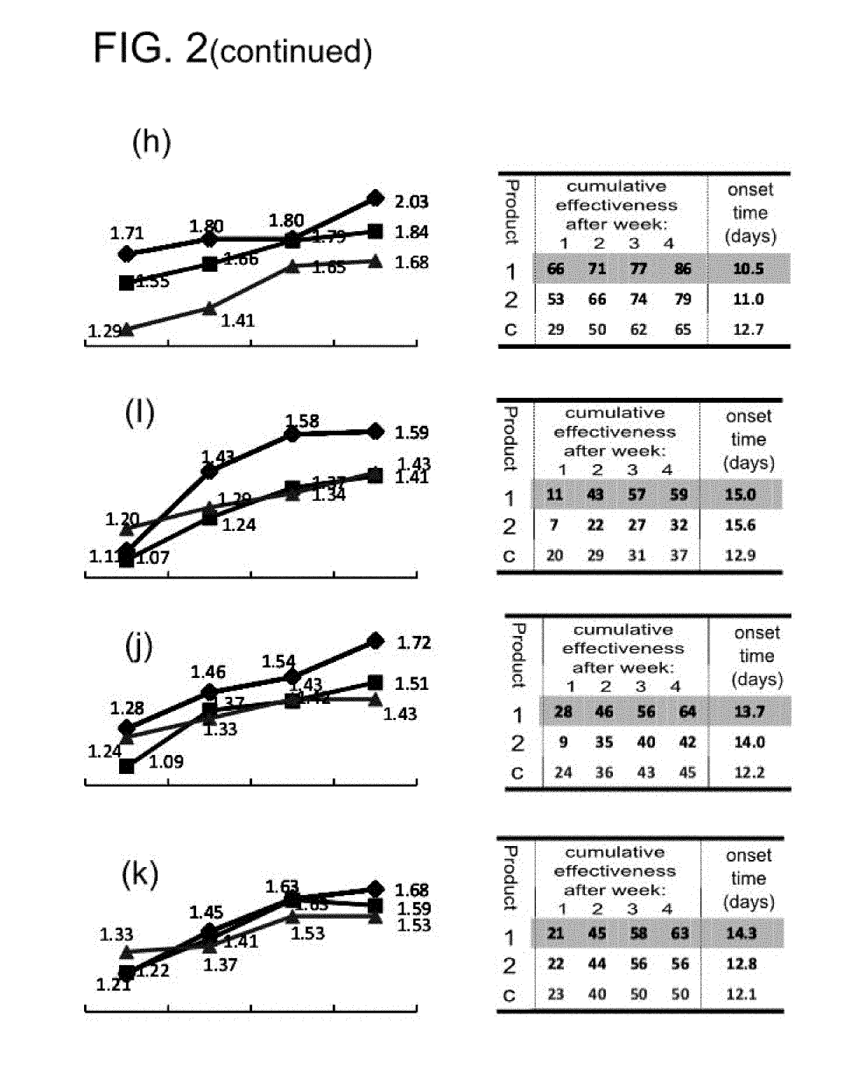

1. Facial wrinkles/fine lines. The result was shown in FIG. 2(a).

2. Rough skin. The result was shown in FIG. 2(b).

3. Dry skin/dehydration. The result was shown in FIG. 2(c).

4. Flabby skin/lack of elasticity. The result was shown in FIG. 2(d).

5. Dull skin tone/lack of gloss. The result was shown in FIG. 2(e).

6. Large skin pore size. The result was shown in FIG. 2(f).

7. Variated skill colors/lack of color uniformity. The result was shown in FIG. 2(g).

8. Oily skin. The result was shown in FIG. 2(h).

9. Skin with colored spots. The result was shown in FIG. 2(i).

10. Pigment sediment. The result was shown in FIG. 2(j).

11. Scar left after acne. The result was shown in FIG. 2(k).

Other skin conditions, such as, reddish-prone skin, couperose-prone skin, scurf-prone skin and allergic sensitive skin, were also tried but the data are not shown here as there were not enough volunteers enrolled for the trial.

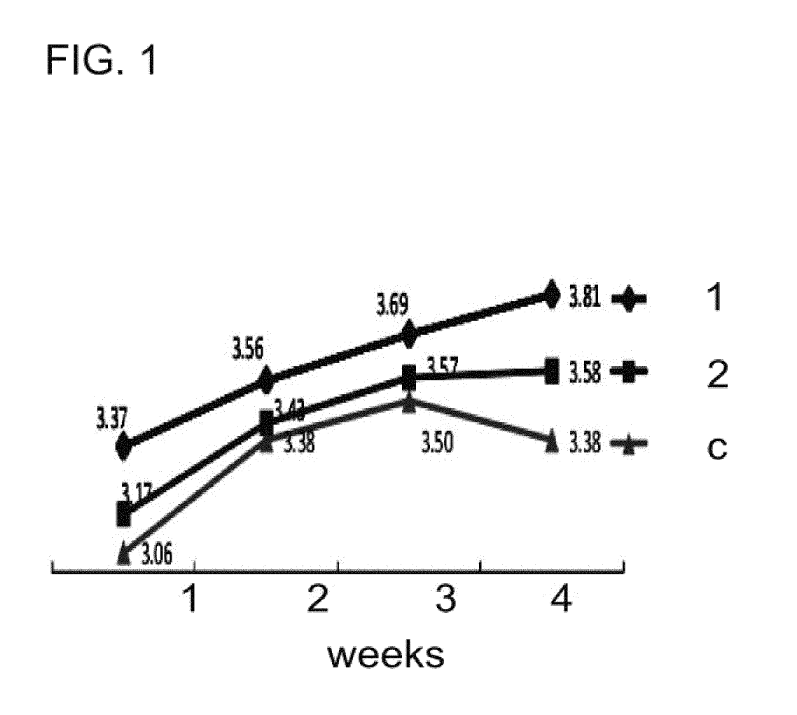

FIG. 1 is the summary of the overall satisfactory rate of each product by the participating volunteers (1, 2 and c refers to products of embodiment 1 embodiment 2 and the comparative prior art product, respectively). As can be seen from FIG. 1, comparing with the existing product available in the art (c), both embodiments (1 and 2) of the present invention demonstrated a superior result. At the end of the 4 week trial, both received higher satisfactory rates, 3.81 and 3.58, respectively, comparing to 3.38 for the prior art product. More significantly, the longer the present invention product was used, the result was better. By comparison, the prior art product peaked at week 3 and continued use had a declined satisfaction. The data suggest that not only the products of the present invention have quicker and better effects from onset but also are better suited for long-term use.

FIG. 2 shows the effects on specific skin conditions/symptoms tried. For example, FIG. 2(a) relates to the condition of facial wrinkles and appearance of fine lines. The left-side curves show that volunteers in the embodiment 1 group (♦) rated the effectiveness at 1.29 (average) after the first week, 1.69 after the second week, 1.79 after the week and 1.90 after the fourth week. The corresponding data for the embodiment 1 group (▪) are 1.31, 1.49, 1.64 and 180 and for the prior art group (▴), the data are 1.23, 1.37, 1.58 and 1.67. For effectiveness rate, 1 means no effect, 2 means some effect, and 3 means significant effects. Understandably, for a cosmetic product, the effectiveness rarely reaches 3. The right-side table shows that Embodiment 1 has an onset time of 13.3 days, which refers to the average time when the volunteers observed at least some effects. The onset time is 13.5 days for Embodiment 2 and 15.3 days for the comparative product. In the Embodiment 1 group, 29% volunteers observed at least some effect (i.e., giving an effectiveness rate of either 2 or 3) at week 1, 62% at week 2, 69% at week 3 and 76% at week 4. The corresponding percentages for Embodiment 2 are 31%, 49%, 58% and 67%. For comparison, the data for the prior art product are 21%, 35%, 53% and 67%. Similar treads can be found in FIG. 2(b)-(k), and it shows that for all the skin conditions/symptoms tried, the products of the present invention had consistently achieved better results than the prior art comparative product in terms of the onset time, the percentage and the degree of effectiveness.

Although not wishing to be bound to any particular theory, the inventors believe that the product of the present invention may achieve its beneficial effects on skin conditions by regulating the immunity and delaying the aging process of skin induced by the sun light. This mechanism was at least part of the consideration in selecting the various ingredients, many of which are derived from natural sources and used in the traditional medicine showing effects in regulating immunity. This may serve as a guideline in modifying the embodiments disclosed herewith and such modifications may achieve similar effects without departing from the spirit of the present invention.

While there have been described and pointed out fundamental novel features of the invention as applied to a preferred embodiment thereof, it will be understood that various omissions and substitutions and changes, in the form and details of the embodiments illustrated, may be made by those skilled in the art without departing from the spirit of the invention. The invention is not limited by the embodiments described above which are presented as examples only but can be modified in various ways within the scope of protection defined by the appended patent claims

What is claimed is:

1. An skin care composition, comprising three part being a powder part, an aqueous part and an emulsion part, each part in a separate containers, wherein:
said powder part comprises the following ingredients in specified portions by weight:

| | |
|---|---|
| Oligopeptide-1 | 0~1.0 portion |
| Ginseng saponin | 0~5.0 portions |

provided that at least one of the ingredients must be present;
said aqueous part comprises the following ingredients in specified portions by weight:

| | |
|---|---|
| Glycyrrhiza glabra Root Extract | 0~3.0 portions |
| Artemisia capillaris Flower Extract | 0~3.0 portions |
| Radix Mori Albae Extract | 0~2.0 portions |
| Zizyphus jujuba Fruit Extract | 0~2.0 portions |
| Scutellaria baicalensis Root Extract | 0~2.0 portions |
| hydrolyzed rice protein | 0~5.0 portions |
| nicotinamide | 0~3.0 portions |

provided that at least one of the ingredients must be present; and
said emulsion part comprises the following ingredients in specified portions by weight:

| | |
|---|---|
| Bifida ferment lysate | 0.5~10.0 portions |
| creatine | 0~2.0 portions |
| carnosine | 0~2.0 portions |
| Glucosyl Hesperidin | 0~5.0 portions |
| Hexapeptide-3 | 0~5.0 portions |
| Centella asiatica Extract | 0~3.0 portions |
| Coenzyme Q10 capsule | 0~2.0 portions |
| Opuntia Ficus-indica Stem Extract | 0~5.0 portions |
| Rhodiola rosea Extract | 0~2.0 portions |
| Saussurea Involucrata Extract | 0~5.0 portions |
| Panax notoginseng Root Extract | 0~5.0 portions |
| Angelica sinensis Extract capsule | 0~5.0 portions |

provided that Bifida ferment lysate and at least one other ingredient must be present.

2. The skin care composition of claim 1, wherein:
said powder part is:

| | |
|---|---|
| Oligopeptide-1 | 0.01~0.5 portion |
| Ginseng saponin | 0.1~2.0 portions. |

3. The skin care composition of claim 2, wherein:
said powder part is:

| | |
|---|---|
| Oligopeptide-1 | 0.01~0.05 portion |
| Ginseng saponin | 0.10~0.50 portion. |

4. The skin care composition of claim 1, wherein:
said aqueous part is:

| | |
|---|---|
| Glycyrrhiza glabra Root Extract | 0.10~1.50 portions |
| Artemisia capillaris Flower Extract | 0.10~1.50 portions |
| Radix Mori Albae Extract | 0.10~1.00 portion |
| Zizyphus jujuba Fruit Extract | 0.10~1.00 portion |
| Scutellaria baicalensis Root Extract | 0.10~1.00 portion |
| hydrolyzed rice protein | 0.10~2.00 portions |
| nicotinamide | 0.10~1.50 portions. |

5. The skin care composition of claim 4, wherein:
said aqueous part is:

| | |
|---|---|
| Glycyrrhiza glabra Root Extract | 0.5~1.0 portion |
| Artemisia capillaris Flower Extract | 0.5~1.0 portion |
| Radix Mori Albae Extract | 0.5~1.0 portion |
| Zizyphus jujuba Fruit Extract | 0.5~1.0 portion |
| Scutellaria baicalensis Root Extract | 0.5~1.0 portion |
| hydrolyzed rice protein | 0.5~1.0 portion |
| nicotinamide | 0.50~1.0 portion. |

6. The skin care composition of claim 1, wherein:
said emulsion part is:

| | |
|---|---|
| Rhodiola rosea Extract | 0.1~2.0 portions |
| Bifida ferment lysate | 0.5~5.0 portions |
| creatine | 0.5~1.5 portions |
| carnosine | 0.1~1.0 portion |
| Glucosyl Hesperidin | 0.1~2.0 portion |
| Hexapeptide-3 | 0.5~1.5 portions |
| Centella asiatica Extract | 0.1~1.0 portion |
| Coenzyme Q10 capsule | 0.1~1.0 portion |
| Opuntia Ficus-indica Stem Extract | 0.1~3.0 portions. |

7. The skin care composition of claim 1, wherein said emulsion is of lotion, gel or cream.

8. The skin care composition of claim 1, wherein said powder part further comprises an external dosage form matrix with one or more ingredients selected from the group consisting of mannitol, disodium hydrogen phosphate and sodium dihydrogen phosphate.

9. The skin care composition of claim 1, wherein said aqueous part further comprises an external dosage form matrix with one or more ingredients selected from the group consisting sodium hyaluronate, oxhide glue, butanediol, disodium EDTA, dipotassium glycyrrhetate, panthenol and preservatives.

10. The skin care composition of claim 7, wherein said emulsion part further comprises an external dosage form matrix with one or more ingredients selected from the group consisting bisabolol, cetylhydroxyproline palmitamide, brassica campestris sterol, jojoba seed oil, phytosterol isostearate, Vitamin E acetate, silicone oil, cetyl stearyl alcohol, sucrose polystearate, Beheneth-25, dipalmitoyl hydroxyproline, Acrylates/C10-30 alkyl acrylate crosspolymer, butanediol, glycerin, sodium stearoyl glutamate, disodium EDTA, xanthan gum, deionized water, acrylamide/ammonia acrylate copolymer-Poly(isobutylene)-Polysorbate-20, 1-methylhydantoin-2-imide and preservatives.
